SEQUENCE LISTING (15 sequences)

SEQ ID NO: 1 - DNA, 5783 bp, Artificial Sequence
  Description of Artificial Sequence pVEX kanR HN-MBP
agatcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaattcacaattctagaaataattttgtttaactttaagaaggagatataaccatgggtcataatcataatcataatcataatcataatcacaacggtggagatgacgatgacaagatgaaaatcgaagaaggaaaagttgttatttggcatgcaatgcaacccaatgagcttgaggtcttccaaagcttagcggaagaatacatggcactctctccagaagttgagatagtttttgaacaaaagccaaacttggaagatgctcttaaggctgcaatacccacaggtcaaggtcctgacctctttatctgggctcacgactggattggaaagtttgctgaggcaggattacttgagccaattgatgaatatgtaactgaagatctccttaacgagtttgctccaatggcccaggatgcaatgcagtataaaggtcactactatgctctaccattcgccgctgaaacagttgcaataatctacaacaaagaaatggttagcgagccaccgaaaacctttgatgagatgaaggcaataatggagaagtactatgatccagcaaatgagaagtatggaatagcttggccaattaatgcctactttatctcagcaattgctcaggcctttggtggttactactttgacgacaaaacagagcaaccgggactagataagcctgagacaatcgaaggatttaagttcttcttcacagaaatatggccatacatggctccaactggagactacaacactcaacagagtatattcctcgagggtagagccccaatgatggttaatggtccatggagcattaacgacgttaagaaggcaggaataaactttggagtggttccactacctccaataatcaaggatggtaaggagtactggccaaggccttacggtggagttaagttgatttacttcgcagcgggaataaagaacaaggatgctgcatggaagttcgcaaagtggcttaccacaagcgaagagtcaattaagacattggcactagagctgggatacataccggttcttacgaaggttcttgatgatccagaaattaagaatgatccagtaatctatggctttggacaagcagttcagcacgcatacctaatgccaaagagtccaaagatgagtgctgtttggggcggagttgatggggcaattaacgaaatcctccaagatccacaaaacgctgacattgaaggaatactaaagaagtatcaacaagaaatccttaacaacatgcaaggaatcgagggaaggcataaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttgccgccgggcgttttttattggtgagaatccaagcactaggcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcga

SEQ ID NO: 2 - DNA, 7285 bp, Artificial Sequence
  Description of Artificial Sequence pVEX kanR HN-MBP-flagellin
agatcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaattcacaattctagaaataattttgtttaactttaagaaggagatataaccatgggtcataatcataatcataatcataatcataatcacaacggtggagatgacgatgacaagatgaaaatcgaagaaggaaaagttgttatttggcatgcaatgcaacccaatgagcttgaggtcttccaaagcttagcggaagaatacatggcactctctccagaagttgagatagtttttgaacaaaagccaaacttggaagatgctcttaaggctgcaatacccacaggtcaaggtcctgacctctttatctgggctcacgactggattggaaagtttgctgaggcaggattacttgagccaattgatgaatatgtaactgaagatctccttaacgagtttgctccaatggcccaggatgcaatgcagtataaaggtcactactatgctctaccattcgccgctgaaacagttgcaataatctacaacaaagaaatggttagcgagccaccgaaaacctttgatgagatgaaggcaataatggagaagtactatgatccagcaaatgagaagtatggaatagcttggccaattaatgcctactttatctcagcaattgctcaggcctttggtggttactactttgacgacaaaacagagcaaccgggactagataagcctgagacaatcgaaggatttaagttcttcttcacagaaatatggccatacatggctccaactggagactacaacactcaacagagtatattcctcgagggtagagccccaatgatggttaatggtccatggagcattaacgacgttaagaaggcaggaataaactttggagtggttccactacctccaataatcaaggatggtaaggagtactggccaaggccttacggtggagttaagttgatttacttcgcagcgggaataaagaacaaggatgctgcatggaagttcgcaaagtggcttaccacaagcgaagagtcaattaagacattggcactagagctgggatacataccggttcttacgaaggttcttgatgatccagaaattaagaatgatccagtaatctatggctttggacaagcagttcagcacgcatacctaatgccaaagagtccaaagatgagtgctgtttggggcggagttgatggggcaattaacgaaatcctccaagatccacaaaacgctgacattgaaggaatactaaagaagtatcaacaagaaatccttaacaacatgcaaggaatcgagggaaggcatatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatatcgatctgaagcagatcaactctcagaccctgggtctggatacgctgaatgtgcaacaaaaatataaggtcagcgatacggctgcaactgttacaggatatgccgatactacgattgctttagacaatagtacttttaaagcctcggctactggtcttggtggtactgaccagaaaattgatggcgatttaaaatttgatgatacgactggaaaatattacgccaaagttaccgttacggggggaactggtaaagatggctattatgaagtttccgttgataagacgaacggtgaggtgactcttgctggcggtgcgacttccccgcttacaggtggactacctgcgacagcaactgaggatgtgaaaaatgtacaagttgcaaatgctgatttgacagaggctaaagccgcattgacagcagcaggtgttaccggcacagcatctgttgttaagatgtcttatactgataataacggtaaaactattgatggtggtttagcagttaaggtaggcgatgattactattctgcaactcaaaataaagatggttccataagtattaatactacgaaatacactgcagatgacggtacatccaaaactgcactaaacaaactgggtggcgcagacggcaaaaccgaagttgtttctattggtggtaaaacttacgctgcaagtaaagccgaaggtcacaactttaaagcacagcctgatctggcggaagcggctgctacaaccaccgaaaacccgctgcagaaaattgatgctgctttggcacaggttgacacgttacgttctgacctgggtgcggtacagaaccgtttcaactccgctattaccaacctgggcaacaccgtaaacaacctgacttctgcccgtagccgtatcgaagattccgactacgcgaccgaagtttccaacatgtctcgcgcgcagattctgcagcaggccggtacctccgttctggcgcaggcgaaccaggttccgcaaaacgtcctctctttactgcgttaactcgaggaagatgggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttgccgccgggcgttttttattggtgagaatccaagcactaggcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcga

SEQ ID NO: 3 - DNA, 6365 bp, Artificial Sequence
  Description of Artificial Sequence pVEX kanR HN-MBP-HA2
agatcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaattcacaattctagaaataattttgtttaactttaagaaggagatataaccatgggtcataatcataatcataatcataatcataatcacaacggtggagatgacgatgacaagatgaaaatcgaagaaggaaaagttgttatttggcatgcaatgcaacccaatgagcttgaggtcttccaaagcttagcggaagaatacatggcactctctccagaagttgagatagtttttgaacaaaagccaaacttggaagatgctcttaaggctgcaatacccacaggtcaaggtcctgacctctttatctgggctcacgactggattggaaagtttgctgaggcaggattacttgagccaattgatgaatatgtaactgaagatctccttaacgagtttgctccaatggcccaggatgcaatgcagtataaaggtcactactatgctctaccattcgccgctgaaacagttgcaataatctacaacaaagaaatggttagcgagccaccgaaaacctttgatgagatgaaggcaataatggagaagtactatgatccagcaaatgagaagtatggaatagcttggccaattaatgcctactttatctcagcaattgctcaggcctttggtggttactactttgacgacaaaacagagcaaccgggactagataagcctgagacaatcgaaggatttaagttcttcttcacagaaatatggccatacatggctccaactggagactacaacactcaacagagtatattcctcgagggtagagccccaatgatggttaatggtccatggagcattaacgacgttaagaaggcaggaataaactttggagtggttccactacctccaataatcaaggatggtaaggagtactggccaaggccttacggtggagttaagttgatttacttcgcagcgggaataaagaacaaggatgctgcatggaagttcgcaaagtggcttaccacaagcgaagagtcaattaagacattggcactagagctgggatacataccggttcttacgaaggttcttgatgatccagaaattaagaatgatccagtaatctatggctttggacaagcagttcagcacgcatacctaatgccaaagagtccaaagatgagtgctgtttggggcggagttgatggggcaattaacgaaatcctccaagatccacaaaacgctgacattgaaggaatactaaagaagtatcaacaagaaatccttaacaacatgcaaggaatcgagggaaggcatatgagaggattatttggagctatagcaggttttatagagggaggatggcagggaatggtagatggttggtatgggtaccaccatagcaatgagcaggggagtgggtacgctgcagacaaagagtctacgcagaaggccatagacggggttaccaacaaggttaattctatcatcgataagatgaacacccaatttgaggctgtgggccgagagttcaataacctggaaagaaggatagagaatctcaacaagaaaatggaggacggtttcctcgacgtgtggacatacaacgccgagcttctcgtgctgatggagaacgaacgcactctagacttccacgactccaatgtcaagaatctttatgacaaggtgcggttgcaacttagagacaatgctaaggagttggggaacggttgttttgagttctaccataagtgtgataatgagtgcatggagtctgttcgaaacgggacgtacgattatccccagtactctgaagaggcccgattgaaaagagaagaaataagtggagtaaaattggaatcaataggaggcaaaaaaaagaaaaagaaagaagaataagaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttgccgccgggcgttttttattggtgagaatccaagcactaggcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcga

SEQ ID NO: 4 - protein, 380 aa, Artificial Sequence
  Description of Artificial Sequence pfMBP
MKIEEGKVVIWHAMQPNELEVFQSLAEEYMALSPEVEIVFEQKPNLEDALKAAIPTGQGPDLFIWAHDWIGKFAEAGLLEPIDEYVTEDLLNEFAPMAQDAMQYKGHYYALPFAAETVAIIYNKEMVSEPPKTFDEMKAIMEKYYDPANEKYGIAWPINAYFISAIAQAFGGYYFDDKTEQPGLDKPETIEGFKFFFTEIWPYMAPTGDYNTQQSIFLEGRAPMMVNGPWSINDVKKAGINFGVVPLPPIIKDGKEYWPRPYGGVKLIYFAAGIKNKDAAWKFAKWLTTSEESIKTLALELGYIPVLTKVLDDPEIKNDPVIYGFGQAVQHAYLMPKSPKMSAVWGGVDGAINEILQDPQNADIEGILKKYQQEILNNMQ

SEQ ID NO: 5 - DNA, 59 bp, Artificial Sequence
  Description of Artificial Sequence PF1938F
tcccagcacctgcacccatgaaaatcgaagaaggaaaagttgttatttggcatgcaatg

SEQ ID NO: 6 - DNA, 56 bp, Artificial Sequence
  Description of Artificial Sequence PF1938R
tcggagcacctgcactaccttccctcgattccttgcatgttgttaaggatttcttg

SEQ ID NO: 7 - DNA, 58 bp, Artificial Sequence
  Description of Artificial Sequence PF1938F2
tcccagcacctgcacccccaaaatcgaagaaggaaaagttgttatttggcatgcaatg

SEQ ID NO: 8 - DNA, 36 bp, Artificial Sequence
  Description of Artificial Sequence fliCF01
ggaaggcatatggcacaagtcattaatacaaacagc

SEQ ID NO: 9 - DNA, 40 bp, Artificial Sequence
  Description of Artificial Sequence fliCR01
ggaagggaattcttaacgcagtaaagagaggacgttttgc

SEQ ID NO: 10 - DNA, 36 bp, Artificial Sequence
  Description of Artificial Sequence HAABEF01
tcccagcatatgagtgatcagatttgcattggttac

SEQ ID NO: 11 - DNA, 46 bp, Artificial Sequence
  Description of Artificial Sequence HACF01
tcccagcatatgagaggattatttggagctatagcaggttttatag

SEQ ID NO: 12 - DNA, 42 bp, Artificial Sequence
  Description of Artificial Sequence HADF01
tcccaacatatgcaccatagcaatgagcaggggagtgggtac

SEQ ID NO: 13 - DNA, 79 bp, Artificial Sequence
  Description of Artificial Sequence HABR01
cgtgagcacctgcaactaattcttattcttctttctttttcttttttttgcccccataccaaccatctaccattccctg

SEQ ID NO: 14 - DNA, 78 bp, Artificial Sequence
  Description of Artificial Sequence HAACDR01
cgtgagcacctgcaactaattcttattcttctttctttttcttttttttgcctcctattgattccaattttactccac

SEQ ID NO: 15 - DNA, 77 bp, Artificial Sequence
  Description of Artificial Sequence HAER01
cgtgagcacctgcaactaattcttattcttctttctttttcttttttttgccctctctttgagggctatttctgagc

(19) United States
(12) Patent Application Publication
(10) Pub. No.: US 2010/0125129 A1
(43) Pub. Date: May 20, 2010

(54) Thermostable Fusion Proteins and Thermostable Adjuvant

(75) Inventor: James A. Williams, Lincoln, NE (US)

Correspondence Address: BROOKS KUSHMAN P.C., 1000 TOWN CENTER, TWENTY-SECOND FLOOR, SOUTHFIELD, MI, 48075, US

(73) Assignee: NATURE TECHNOLOGY CORP., Lincoln, NE (US)

(21) Appl. No.: 12/621,960

(22) Filed: Nov. 19, 2009

Related U.S. Application Data
(60) Provisional application No. 61/199,651, filed on Nov. 19, 2008.

(51) Int. Cl. C07K 14/00; C12P 21/06

(52) U.S. Cl. 530/350; 435/69.7

(57) ABSTRACT

Fusion proteins including maltodextrin-binding protein (MBP) domains that are thermally stable and soluble are provided. Methods for forming and using the fusion proteins are also provided.

### CROSS REFERENCE TO RELATED APPLICATIONS

The application claims the benefit of provisional application Ser. No. 61/199,651, filed Nov. 19, 2008, the disclosure of which is hereby incorporated in its entirety.

### STATEMENT REGARDING FEDERALLY SPONSORED RESEARCH OR DEVELOPMENT

This invention was made in part with government support under Grant No. 2 R44 GM072141-02, awarded by the National Institutes of Health. The government has certain rights in this invention.

### SEQUENCE LISTING

The text file Thermostable Fusion Proteins Oct09_ST25 with a Date Created of Nov. 19, 2009 and a size of 32000 Bytes, filed herewith, is hereby incorporated by reference.

### FIELD OF THE INVENTION

The present invention relates to the production of recombinant proteins, and more particularly, is a method to improve thermostability of said molecules for application as adjuvants or antigens.

### BACKGROUND OF THE INVENTION

Recombinant proteins are useful in therapy, diagnostics, biotechnology, preventive vaccination, therapeutic vaccination, cancer vaccination, and agricultural vaccination.

Recombinant protein thermostability can be improved by fusion to thermostable proteins (de Marco A, Casatta E, Savaresi S, Geerlof A, 2004, J. Biotechnol. 107: 125-133). However, the system utilized (fusion to thermostable Methanopyrus kandleri Ftr protein) only improved thermostability with small target proteins of <20 kd. The authors teach that thermo-stabilization requires a minimal ratio of about 2 between the molecular weights of the thermostable carrier partner and the target protein (2:1 carrier:target protein size ratio). This severely limits application of thermostable fusion partners to only small target proteins.

Thermostable archaea maltodextrin-binding proteins (MBP), such as Pyrococcus furiosus maltodextrin-binding protein (pfMBP), have been shown to confer solubilization properties onto fusion proteins (Fox J D, Routzahn K M, Bucher M H, Waugh D S. 2003 FEBS Lett 537:53-57). However, this study did not assess the ability of archaea MBPs to improve thermostability of target proteins.

pfMBP (43 kd) carrier protein was subsequently shown to improve thermostabilization of a small (27 kd) target fluorescent GFP protein (Huang H, Liu J, deMarco A. Biochem. Biophys. Res. Commun. 344: 25-29). The overall size of this fusion protein is consistent with the estimated requirements of approximately 2:1 thermostable carrier partner to target protein size ratio of de Marco et al, Supra, 2004.

Accordingly, there is a need for a thermo-stabilization fusion partner that generically improves target protein thermostability, even with large target proteins exceeding the size of the thermostable fusion partner.

### SUMMARY OF THE INVENTION

The present invention solves one or more problems of the prior art by providing in at least one embodiment a method for improving thermostability of a target protein. The target protein is expressed as a fusion with thermostable maltodextrin-binding protein (MBP). Fusion to MBP thermo-stabilizes the target protein. The invention relates to methods for producing thermostable recombinant fusion proteins using a bacterial cell (such as the gram negative bacterium E. coli) as a production host. Thermostable fusion protein embodiments of the invention provide for a rational approach to improve thermostability of a target protein.

In at least one aspect, the present invention provides a thermostable MBP that can be utilized as a generic thermostabilization tag. Surprisingly, some embodiments of the invention provide stabilization with much greater amounts of target protein than taught by De Marco et al, Supra, 2004.

In another aspect of the present invention, a thermostable adjuvant produced using this system is also disclosed. A novel thermostable antigen produced using this system is also disclosed. Vectors for producing the thermostable fusion proteins in a bacterial host (E. coli) are also disclosed. However, the thermostable fusion proteins of the invention could be produced in other bacterial hosts, or in eukaryotic cells, utilizing an alternative expression system described in the art.

In another aspect of the present invention, a fusion protein that elicits an immunological response is provided. In this regard, the fusion proteins retain or enhance the antigenicity of the target protein, while enabling production through improved solubility. Thermostable vaccines utilizing these fusion proteins potentially reduce storage costs for animal (including human) vaccines. Moreover, vaccines composed of thermostable antigens, or combinations of thermostable adjuvants and thermostable antigens, would be valuable for combat zone military application since cold storage to maintain activity is impractical for deployed soldiers. Thermostable diagnostic reagents and therapeutics that do not require cold storage would also find application to reduce cost and enable utilization for applications when cold storage is impractical.

Other exemplary embodiments of the invention will become apparent from the detailed description provided hereinafter. It should be understood that the detailed description and specific examples, while disclosing exemplary embodiments of the invention, are intended for purposes of illustration only and are not intended to limit the scope of the invention.

### BRIEF DESCRIPTION OF THE DRAWINGS

FIG. 1. is a map showing Hemagglutinin fusion proteins shows influenza serotype H5 hemagglutinin protein, with the locations of the HA regions expressed as MBP-HA fusion proteins.

FIG. 2 pVEX kanR HN-MBP

FIG. 3 is a map pVEX kanR HN-MBP-flagellin

FIG. 4 is a map pVEX kanR HN-MBP-HA2

### DESCRIPTION OF THE SEQUENCES

SEQ ID NO:1 pVEX kanR HN-MBP

SEQ ID NO:2 pVEX kanR HN-MBP-flagellin

SEQ ID NO:3 pVEX kanR HN-MBP-HA2

SEQ ID NO:4 pfMBP

SEQ ID NO:5 PF1938F

SEQ ID NO:6 PF1938R

SEQ ID NO:7 PF1938F2

SEQ ID NO:8 fliCF01

SEQ ID NO:9 fliCR01

SEQ ID NO:10 HAABEF01

SEQ ID NO:11 HACF01

SEQ ID NO:12 HADF01

SEQ ID NO:13 HABR01

SEQ ID NO:14 HAACDR01

SEQ ID NO:15 HAER01

### DETAILED DESCRIPTION OF THE INVENTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. The description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

Throughout this application, where publications are referenced, the disclosures of these publications in their entireties are hereby incorporated by reference into this application to more fully describe the state of the art to which this invention pertains.

Definitions

MBP: Maltodextrin-binding protein

bp: basepairs

His-tag: 6-10 amino acid metal binding sequence (histidine=H) 6-10× that binds to metal chelate affinity resins similarly to the HN-tag sequence

HN-tag: 12 amino acid metal binding sequence (histidine-asparagine=HN) 6× that binds to metal chelate affinity resins similarly to the His-tag sequence

IPTG: isopropyl-beta-D-thiogalactopyranoside

mM: milliMolar

OD₆₀₀: optical density at 600 nm wavelength of light.

PCR: Polymerase Chain Reaction

pfMBP: Maltodextrin-binding protein from Pyrococcus furiosus

TLR5=Toll-like receptor 5

The term “protein adjuvant” as used herein refers to a protein or protein domain that increases the antigenic response.

The term “operably linked” as used herein, means a functional linkage between the expression control sequence and the coding sequence to which it is linked. The operable linkage permits the expression control sequence to control expression of the coding sequence. Expression control sequences can include a promoter, a transcriptional activator binding sequence, an enhancer sequence or any other regulatory or non-regulatory sequence that may be required for transcription and translation of the coding sequence to which the expression control sequence is linked.

In an embodiment of the present invention, a fusion protein with improved thermal stability is provided. The fusion protein of this embodiment includes a maltodextrin-binding protein domain and a target protein domain. In a refinement, the target protein domain is of a sufficient molecular weight such that at least 50% of fusion protein is the target protein domain. In another refinement, the target protein domain is of a sufficient molecular weight such that at least 60% of fusion protein is the target protein domain. In still another refinement, the target protein domain is of a sufficient molecular weight such that at least 70% of fusion protein is the target protein domain. In yet another refinement, the target protein domain is of a sufficient molecular weight such that at least 80% of fusion protein is the target protein domain. Advantageously, the fusion protein possesses improved thermostability when compared to a protein which only includes the target protein domain and not the maltodextrin-binding protein domain. In one preferred embodiment, the thermostable MBP is used in a fusion protein to improve thermostability of a target protein. Thermostable MBP fusions have application to redesign traditionally thermolabile proteins for improved yield, solubility, stability and thermostability. This has application in vaccination, therapy, diagnostics, or other applications where improved protein stability is desirable.

In a variation of the present embodiment, the maltodextrin-binding protein domain has an amino acid sequence that is at least 80 percent identical to the amino acid sequence set forth as SEQ ID NO: 4. In other refinements, the maltodextrin-binding protein domain has an amino acid sequence that is at least, in order of increasing preference, 85%, 90%. 91%, 92%, 93%, 94%, 95%, 96%, 97%, 98%, 98.5% or 99% percent identical to the amino acid sequence set forth as SEQ ID NO: 4. In one embodiment, a robust thermostabilization methodology that incorporates one or more thermostable Maltodextrin-binding proteins (“MBPs”) as (a) fusion carrier(s) in order to stabilize target proteins is provided. The invention may be practiced to engineer thermostable varieties of otherwise thermolabile protein vaccines and therapeutics. This practice eliminates complex protein mutagenesis methodologies that are currently utilized to redesign thermolabile protein vaccines and therapeutics. These prior methods are by nature time consuming, complex, and difficult, and success is unpredictable, compared to the instant invention. In a variation of the present embodiment, the thermostable MBP is an archaea MBP. In a refinement, the archaea MBP is pfMBP. In a further refinement, target proteins are expressed C-terminal to native pfMBP. In another refinement, the target proteins are expressed C-terminal to pfMBP, that optionally contains a further N terminal purification tag. In yet another refinement, the N terminal purification tag is a his-tag. In still another refinement, the N terminal purification tag is a HN-tag.

In another variation of the present embodiment, the target protein domain is derived from a protein selected from the group consisting of: viral pathogen antigens; bacterial pathogen antigens; influenza antigens; influenza hemagglutinin; influenza hemagglutinin HA1 domain; influenza hemagglutinin HA2 domain; protein adjuvants; and flagellin. In the present context, “derived from” means that the domain is in order of increasing preference 85%, 90%. 91%, 92%, 93%, 94%, 95%, 96%, 97%, 98%, 98.5% or 99% percent identical to the amino acid sequence of a protein in the group.

In another embodiment of the present invention, a method for improving the thermostability of a target protein molecule is provided. In accordance with the method of the present embodiment, a DNA expression vector which expresses a fusion protein in a transformed host cell is constructed. The expression vector is then introduced into a host cell that subsequently expresses the fusion protein therein. The fusion protein is then recovered and optionally purified. Advantageously, the fusion protein possesses improved thermostability when compared to a protein which only includes the target protein domain and not the maltodextrin-binding protein domain.

In another embodiment of the present invention, a fusion protein with sufficient solubility to function as an immunogen is provided. The fusion protein of this embodiment includes a maltodextrin-binding protein domain and a target protein antigen domain. In a refinement, the target protein antigen domain is of a sufficient molecular weight such that at least 50% of fusion protein is the target protein domain. In another refinement, the target protein domain is of a sufficient molecular weight such that at least 60% of fusion protein is the target protein domain. In still another refinement, the target protein antigen domain is of a sufficient molecular weight such that at least 70% of fusion protein is the target protein domain. In yet another refinement, the target protein antigen domain is of a sufficient molecular weight such that at least 80% of fusion protein is the target protein domain. Examples of target protein antigen domains are derived from protein antigens selected from the group consisting of: viral pathogen antigens; bacterial pathogen antigens; influenza antigens; influenza hemagglutinin; influenza hemagglutinin HA1 domain; and influenza hemagglutinin HA2 domain. In the present context, “derived from” means that the domain is in order of increasing preference 85%, 90%. 91%, 92%, 93%, 94%, 95%, 96%, 97%, 98%, 98.5% or 99% percent identical to the amino acid sequence of the protein antigens. In the present embodiment, the target protein fusion with MBP retains or enhances the antigenicity of the target protein, while enabling production through improving solubility. In a variation of the present invention, the purified fusion protein is administered to an animal (including a human) to elicit an immunological response.

In an analogous manner as set forth above, the maltodextrin-binding protein domain of the present embodiment has an amino acid sequence that is at least 80 percent identical to the amino acid sequence set forth as SEQ ID NO: 4. In other refinements, the maltodextrin-binding protein domain has an amino acid sequence that is at least, in order of increasing preference, 85%, 90%. 91%, 92%, 93%, 94%, 95%, 96%, 97%, 98%, 98.5% or 99% percent identical to the amino acid sequence set forth as SEQ ID NO: 4.

In another embodiment of the present invention, a method for producing a target protein antigen as a fusion protein is provided. The details of the target protein antigen are set forth above. In accordance with the present embodiment, a DNA expression vector which expresses a fusion protein in a transformed host cell is constructed. The fusion protein includes a maltodextrin-binding protein domain and a target protein domain. The expression vector is introduced into a host cell that expresses the fusion protein. The fusion protein is then recovered. Advantageously, in a variation of the present invention, the recovered protein is then introduced into a subject wherein an immune response is elicited.

In still another embodiment of the present invention, a thermostable protein adjuvant-containing fusion protein is provided. The thermostable protein adjuvant-containing fusion protein includes a maltodextrin-binding protein domain and a protein adjuvant domain. Advantageously, the fusion protein possesses improved thermostability when compared to a protein which only includes the target protein domain and not the maltodextrin-binding protein domain. In a refinement, the target protein adjuvant domain is of a sufficient molecular weight such that at least 50% of fusion protein is the target protein domain. In another refinement, the target protein adjuvant domain is of a sufficient molecular weight such that at least 60% of fusion protein is the target protein domain. In still another refinement, the target protein adjuvant domain is of a sufficient molecular weight such that at least 70% of fusion protein is the target protein domain. In yet another refinement, the target adjuvant protein domain is of a sufficient molecular weight such that at least 80% of fusion protein is the target protein domain. In a variation of the present invention, the protein adjuvant domain is derived from a protein selected from the group consisting of: toll-like receptor agonist; complement receptor agonist; flagellin; salmonella flagellin; porin; Neisserial porin; Neisseria meningitidis PorB; type 1 fimbria fimH adhesion protein; cholera ADP-ribosylating enterotoxin; Escherichia coli heat labile enterotoxin; HSP60; cytokines; chemokines; and Toxoplasma gondii profilin. In the present context, “derived from” means that the domain is in order of increasing preference 85%, 90%. 91%, 92%, 93%, 94%, 95%, 96%, 97%, 98%, 98.5% or 99% percent identical to the amino acid sequence of a protein in the group.

In yet another embodiment of the present invention, a method of forming a thermostable protein adjuvant-containing fusion protein is provided. In accordance with the method of the present embodiment, a DNA expression vector which expresses the thermostable protein adjuvant-containing fusion protein in a transformed host cell is constructed. The expression vector is then introduced into a host cell that subsequently expresses the fusion protein therein. The fusion protein is then recovered and purified. Advantageously, the fusion protein possesses improved thermostability when compared to a protein which only includes the target protein domain and not the maltodextrin-binding protein domain. In this embodiment, the thermostable MBP is used to create a thermostable adjuvant. In one refinement, the thermostable adjuvant is pfMBP-flagellin. Thermostable adjuvants have application as adjuvant for live virus, attenuated virus, live bacterial, attenuated bacterial, inactivated bacterial, inactivated viral, protein or DNA vaccination for treatment of bacterial or viral infectious diseases, cancer, autoimmune disorders, misfolded protein disease (e.g. Alzheimer's, Prion disease), allergy, etc.

The embodiments set forth above each utilize an amino acid sequence derived from SEQ ID No. 4. The amino acid sequence SEQ ID No. 4 is a fusion protein partner that is used to improve target protein production yields, target protein extraction, target protein purification, target protein thermostability and target protein applications such as vaccination. A proteolytic cleavage site may optionally be included between the fusion protein partner and the target protein sequence of interest to allow removal of fusion protein sequences from the target protein. Examples of other fusion protein partners include glutathione-S-transferase (GST), 6× Histidine, thioredoxin and β-galactosidase. A review of E. coli fusion partner expression systems is provided in LaVallie E R, McCoy J M. Curr Opin Biotechnol. 1995; 6:501-506, which is hereby incorporated by reference.

The embodiments set forth above relate to SEQ ID No. 4 or a variant or derivative or fragment thereof. Thus, the present invention also encompasses the use of sequences having a degree of sequence identity with the SEQ ID No. 4 amino acid sequence. Herein, the term “sequence identity” means a polypeptide having a certain similarity with the subject amino acid sequence.

Thus, certain embodiments also encompass the use of variants, homologues and derivatives of any amino acid sequence of a protein as defined herein, particularly those of subject polypeptide sequence SEQ ID No. 4.

The similar amino acid sequence should provide and/or encode a polypeptide which retains the functional activity of the sequence. A similar sequence includes an amino acid sequence is at least, in order of increasing preference, 85%, 90%. 91%, 92%, 93%, 94%, 95%, 96%, 97%, 98%, 98.5% or 99%, identical to the subject polypeptide sequence.

In other variations of the present invention, it is preferred to express sequence similarity in terms of sequence identity (i.e. identical amino acid residues) rather than similarity (i.e. amino acid residues having similar chemical properties). Sequence identity comparisons can be conducted using sequence comparison computer programs that use algorithms to align two or more sequences using a scoring system that rewards alignment of identical or similar amino acids and penalizes substitutions of non-similar amino acids and gaps. Computer programs for carrying out alignments include, but are not limited to, BLASTP [from the BLAST (Basic Local Alignment Search Tool that is publicly available from NCBI (http://www.ncbi.nlm.nih.gov/)] and Vector NTI (Invitrogen Corp.). These programs calculate percent sequence identity and report the determined value. It is preferred to use the default values when using such software for amino acid sequence alignments. BLASTP is preferred to determine amino acid sequence identity between the subject polypeptide sequence SEQ ID No. 4 and a candidate polypeptide sequence according to the present invention. Details of the BLASTP algorithm are set forth in D. W. Mount “Bioinformatics: Sequence and Genome Analysis.”, Cold Spring Harbor Press (2004). A particularly preferred set of parameters for the BlastP alignment (which should be used unless otherwise specified) are a Blossum 62 scoring matrix with a gap penalty of 11, a gap extend penalty of 1, and conditional adjustments set to conditional compositional score matrix adjustment. Other preferred parameters for the BlastP alignment are an expect threshold of 10 and s word size of 3.

The sequences, particularly those of subject polypeptide SEQ ID No. 4, may also have deletions, insertions or substitutions of amino acid residues which result in a functionally equivalent protein. This includes rationally designed amino acid substitutions made on the basis of similarity in polarity, charge, solubility, hydrophobicity, and/or the amphipathic nature of the residues, as long as the relevant function is retained. Conservative substitutions that may be made are, for example, substitutions of aliphatic amino acids (Alanine, Valine, Leucine, Isoleucine), polar amino acids (Glutamine, Asparagine, Serine, Threonine), acidic amino acids (glutamic acid and aspartic acid), basic amino acids (Arginine, Lysine and Histidine), aromatic amino acids (Phenylalanine, Tryptophan and Tyrosine), large amino acids (Phenylalanine and Tryptophan), small amino acids (Glycine, Alanine) and hydroxyl amino acids (Serine, Threonine).

As set forth above, some embodiments of the invention include expression vectors that include a nucleic acid encoding the fusion proteins described herein. The term “expression vector” refers to a nucleic acid molecule capable of transporting another nucleic acid to which it has been linked. In one refinement, the vector is capable of autonomous replication. In another variation, the vector integrates into a host DNA. Those skilled in the art of molecular biology will readily recognize that a number of expression vectors are successfully used in the present embodiment. An expression vector contains functional components required for the production of a protein of interest. This includes a suitable RNA polymerase promoter to direct transcription of the gene of interest; transcription termination sequences after the gene of interest to terminate transcription; and translation initiation sequences prior to the gene of interest to promote translation of the gene of interest. An expression vector according to the invention may be constructed by methods known in the art, for example as described in Makrides S C, 1996, Microbiol. Rev. 60:512-538 which is hereby incorporated by reference in its entirety. Examples of useful expression vectors include, but are not limited to, plasmid vectors and viral vectors. Specific examples of viral vectors include, but are not limited to, vectors derived from pox viruses, retroviruses, SV40 virus, adenovirus, adeno-associated virus, or herpes viruses. Once introduced into a host cell, the vector can remain episomal or may become chromosomal (i.e., incorporated into the genome of the host cell). Examples of host cells include, but are not limited to, bacterial cells, yeast cells, insect cells, avian cells, or mammalian cells, and the like. A vector can include the fusion protein encoding nucleic acid in a form suitable for expression of the nucleic acid in a host cell. Typically, the expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce the fusion proteins set forth above.

In yet another embodiment of the present invention, nucleic acid sequences encoding the fusion proteins set forth above (including fusion proteins with conservative substitutions therein) are provide (i.e., one nucleic acid sequence for each fusion protein).

The method of the invention is further illustrated in the following examples. These are provided by way of illustration and are not intended in any way to limit the scope of the invention.

### Examples

### Example 1

### Development of a Thermostable Fusion Protein pfMBP Based Expression System

The MBP gene (PF1938) from Pyrococcus furiosus DSM 3638 (ATCC 43587D-5) was PCR amplified from genomic DNA using the following primer pairs to make cytoplasmic (1) or periplasmic (2) expression constructs. All clones were sequence verified.

1) (pfMBP) (MKIEE-MBP-GIEGR LINKER)

SEQ ID NO: 5

PF1938F:
TcccagcacctgcacccATGAAAATCGAAGAAGGAAAAGTTGTTATTTGG
CATGCAATG

Underlined are substitutions at the 5′ end to delete the pfMBP secretion leader and replace the 5′ end with MKIEE amino acids (residues 1-5 of SEQ ID NO: 4).

SEQ ID NO: 6

PF1938R:
tcggagcacctgcactaCCTTccctcgatTCCTTGCATGTTGTTAAGGAT
TTCTTG

Double underlined is Glycine amino acid, single underlined is the thrombin cleavage linker. This facilitates removal of MBP from downstream fusions if desired. The cloning site is MBP-gga atc gag gga agg cat atg (residues 1491-1511 of SEQ ID NO:3) (CATATG is NdeI cloning site for the start of the downstream fusion protein).

The 1.2 kb product was digested with AarI (recognizes cacctgc and cleaves +4 and +8 by 3′ to the recognition sequence) and cloned into the pVEXB expression vector backbone (Disclosed in Williams J A, and Hodgson C P. 2006. WO/2006/026125).

2) (pfMBP gly ser) (MKIEE-MBP-GGGGGS LINKER)

A second version, with the MBP linker modified to encode 5 glycine residues and a serine residue immediately before the CATATG NdeI cloning site for the target protein, was constructed by PCR mediated mutagenesis of the clone above.

3) (pfMBP PERIPLASMIC) (OMPA-KIEE-MBP-GIEGR LINKER

SEQ ID NO: 7

PF1938F2:
tcccagcacctgcacccCCAAAATCGAAGAAGGAAAAGTTGTTATTTGGC
ATGCAATG

Underlined are substitution at the 5′ end to fuse to the OmpA secretion leader and replace the 5′ end of MBP (secretion tag and start codon) with KIEE amino acids (same as E. coli) immediately downstream of OmpA.

SEQ ID NO: 6

PF1938R:
tcggagcacctgcactaCCTTccctcgatTCCTTGCATGTTGTTAAGGAT
TTCTTG

Double underlined is Glycine amino acid, single underlined is the factor Xa cleavage linker. This facilitates removal of MBP from downstream target proteins if desired. This cleavage site may be substituted with other site specific protease recognition sequences known in the art, such as enterokinase, TEV, PreScission, etc.

The 1.2 kb product was digested with AarI and cloned into the pVEXBOmpA expression vector backbone (Disclosed in Williams J A, and Hodgson C P. 2006. WO/2006/026125).

This vector secretes MBP periplasmically using the OmpA leader, and may be useful for expression of fusion proteins that are toxic when expressed cytoplasmically.

4) (pfHNMBP) (M HN-TAG KIEE-MBP-GIEGR LINKER)

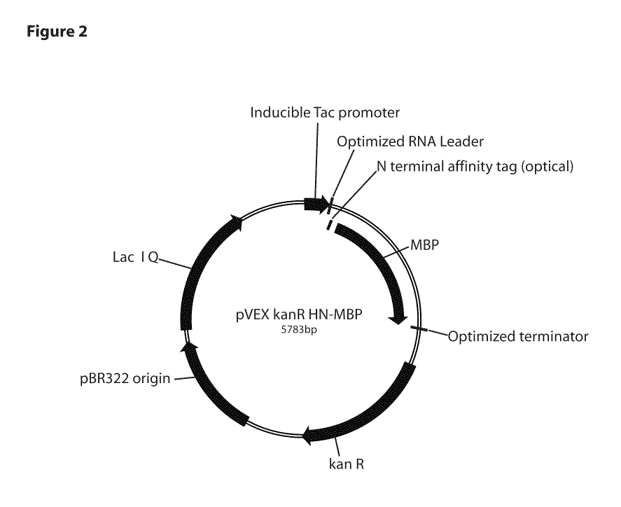

A HN tagged derivative of pfMBP was constructed by modification of pfMBP above. The map (FIG. 2.) and sequence of one of these plasmids pVEX Kan HN MBP, is shown in SEQ ID NO: 1. This vector is a kanamycin resistant production plasmid, in which pfMBP expression is controlled by the tac promoter.

### Example 2

### Thermostable Adjuvant

A fusion of pfMBP (43 kd) and flagellin (52 kd) was made in heat inducible (pR) and arabinose inducible (AraB) versions of the pfMBP and the pfMBP-glyser vectors from above. The fliC (flagellin) gene from Salmonella enterica serovar typhimurium (ATCC genomic DNA 700720D) was PCR amplified with following primers.

SEQ ID NO: 8

fliCF01:
ggaaggcatatggcacaagtcattaatacaaacagc

SEQ ID NO: 9

fliCR01:
ggaagggaattcttaacgcagtaaagagaggacgttttgc

The 1.5 kb PCR product was digested with NdeI/EcoRI (sites are underlined in the primers) and cloned into the pfMBP and the pfMBP-glyser vectors. All clones were sequence verified.

Thermostable fusion protein (75° C. for 20 minutes) of the correct size was produced after heat induction of the pR clones, or arabinose induction of the araB clones. Native E. coli proteins were precipitated with this heat treatment. This demonstrates that fusions of pfMBP and flagellin are proteolytically and thermally stable and that this stability is conferred by two different linkers and can be expressed by at least two different promoters. This also demonstrates the novel observation that pfMBP (43 kd) can stabilize a much larger target protein (52 kd flagellin; 0.8:1 carrier:target protein size ratio), contrary to the teachings of de Marco et al., Supra, 2004 that a 2:1 carrier:target protein size ratio is required for thermostabilization.

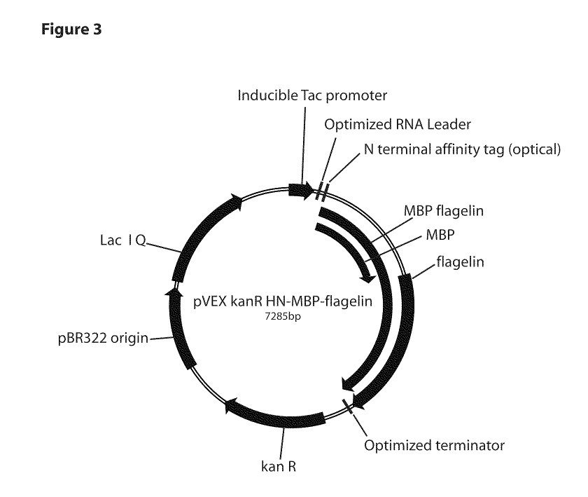

The flagellin gene was also cloned in the pVEX kan HN MBP vector. The map (FIG. 3) and sequence of the resultant clone, pVEXkan HN MBP-flagellin is shown in SEQ ID NO: 2. High levels of thermostable fusion protein were induced after IPTG induction of the tac promoter. This demonstrates that addition of the 2.3 kd N terminal HN tag to pfMBP does not affect expression or thermostability of the composite fusion protein.

Thermostable fusion protein was purified from a 1 Liter IPTG induced shake flask culture. Cells were sequentially: resuspended in binding buffer (50 mM NaPO₄, pH 7, 300 mM NaCl); lysed by freeze thaw and sonication; and centrifuged. E. coli proteins were precipitated by heat treatment (75° C. for 40 minutes). The clarified lysate (primarily a single HN-MBP-Flagellin protein of the expected size) was added to a Talon resin (BD Biosciences) column, and the adherent protein was washed in binding buffer containing 0.1% Triton X-114 (4° C.), to reduce endotoxin. Adherent protein was eluted in elution buffer (binding buffer containing 200 mM imidazole). The final yield was 66 mg of high purity, intact, low endotoxin fusion protein (<26 EU/mg). This demonstrated that the pVEXkan HN MBP-Flagellin expression system can be utilized to produce large amounts of high quality Flagellin protein. A control purification from 1 liter shake flask culture of HNMBP using the methodology described above yielded 92 mg highly pure low endotoxin protein (20 EU/mg).

The immunostimulatory activity of the low endotoxin preparation of fusion proteins HN MBP-Flagellin, HN MBP (negative control) and recombinant flagellin (Invivogen, San Diego, Calif.) was determined in cell culture. HEK 293 cells in 24 well plates were transfected with either: 1) pUNO-TLR9 (Invivogen; CpG DNA responsive TLR); or 2) pUNO-TLR5 (Invivogen; Flagellin responsive TLR). Both TLR9 and TLR5 wells were co-transfected with NFκB-luc (luciferase reporter plasmid responsive to TLR mediated NFκB activation). 0.2 or 2 μg of each protein was added per well 20 hrs post transfection and luciferase activity determined 6 hrs later. Strong activation was observed with TLR5, but not TLR9, with both doses of recombinant flagellin and HN MBP-Flagellin but not with either dose of HN MBP. TLR5 activation activity was not reduced with HN MBP-Flagellin preparations that were further stressed by 3× freeze-thaw or additional heat treatment (71° C. 1 hr). This demonstrates that the pfMBP-Flagellin fusion combines thermostability with immunostimulatory activation of TLR5 and that the pVEX HNpfMBP-Flagellin expression system can be utilized to produce large amounts of high quality thermostable Flagellin protein that is compatible with standard formulations and freeze thaw. pfMBP could be applied to stabilize other immunostimulatory proteins, exemplified in a non-limiting list including: toll-like receptor 2 activating Neisseria meningitidis PorB; toll-like receptor 4 agonist type 1 fimbria fimH adhesion protein; ADP-ribosylating enterotoxins (e.g. cholera toxin and Escherichia coli heat labile enterotoxin); HSP60; cytokines; chemokines; and Toxoplasma gondii profilin.

### Example 3

### pfMBP Vectors and HA Fusions

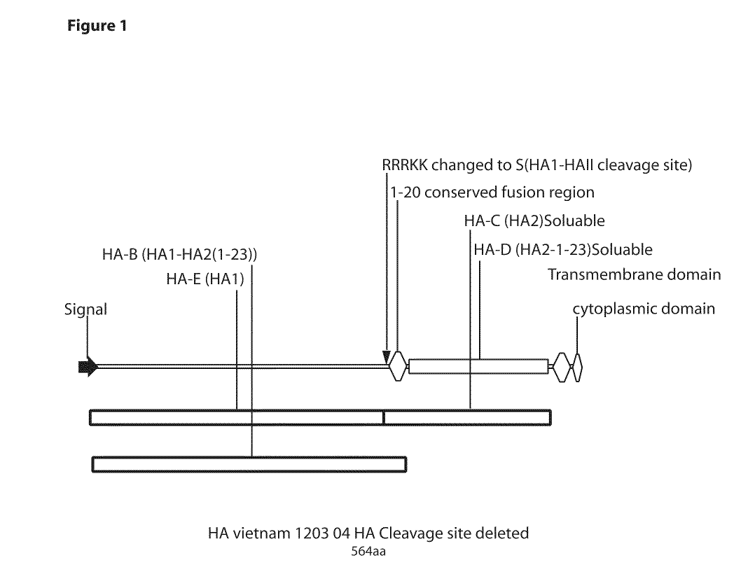

An arabinose-inducible version of the pfMBP expression vector described above (pVEXBMBP) was utilized to construct a series of four fusions (B to E in Table 1 below) with the influenza H5 vietnam 1203 04 serotype hemagglutinin (HA) gene using the fragments described below in Table 1 and shown in FIG. 1.

TABLE 1
pfMBP fusion proteins

| | | PCR | | MBP:Target |
|---|---|---|---|---|
| Target | | product | Fusion | protein size |
| Protein | Primers | size | Protein size | ratio* |
| B = | HAABEF01 + | 1.1 kb | 85 kd | 1:1 |
| HA1-HA2 1- | HABR01 | | | |
| 23 | | | | |
| C = HA2 | HACF01 + | 0.6 kb | 66 kd | 1.9:1 |
| | HAACDR01 | | | |
| D = HA2 (-1- | HADF01 | 0.5 kb | 63 kd | 2.2:1 |
| 23) | HAACDR01 | | | |
| E = HA1 | HAABEF01 + | 1.0 kb | 82 kd | 1.1:1 |
| | HAER01 | | | |
| Flagellin | fliCF01 + | 1.5 kb | 95 kd | 0.8:1 |
| (example 2) | fliCR01 | | | |

*MBP carrier protein = 43 kd

Forward Primers:

All clones deleted the N terminal signal peptide and C terminal transmembrane domain-cytoplasmic domain. 1-23 of HA2 is the hydrophobic membrane insertion region that is conserved across multiple isolates.

SEQ ID NO: 10

HAABEF01:
tcccagCATATGagtgatcagatttgcattggttac

SEQ ID NO: 11

HACF01:
tcccagCATATGagaggattatttggagctatagcaggttttatag

SEQ ID NO: 12

HADF01:
tcccaaCATATGcaccatagcaatgagcaggggagtgggtac

NdeI (CATATG) on forward primers is compatible and in frame with NdeI following the MBP in both clones

Reverse Primers:

All clones used AarI to make EcoRI compatible end, at the 3′ end of the gene, and add a eight amino acid sequence composed of glycine-six lysine residues and two glutamic acid residues (underlined) to the C-terminal end of each flu protein. Stop codon is double underlined.

SEQ ID NO: 13

HABR01:
cgtgagcacctgcaactAATTCTTATTCTTCTTTCTTTTTCTTTTTTTTG
CCCCCATACCAACCATCTACCATTCCCTG

SEQ ID NO: 14

HAACDR01:
cgtgagcacctgcaactAATTCTTATTCTTCTTTCTTTTTCTTTTTTTTG
CCTCCTATTGATTCCAATTTTACTCCAC

SEQ ID NO: 15

HAER01:
cgtgagcacctgcaactAATTCTTATTCTTCTTTCTTTTTCTTTTTTTTG
CCCTCTCTTTGAGGGCTATTTCTGAGC

All clones were sequence verified and expressed in bacterial cells after arabinose induction.

All four pfMBP fusions, HA1-HA2 (1-23), HA2, HA2 (−1-23) and HAL were: stable; soluble in TE when lysed in small scale cultures; and thermostable after heat treatment. This demonstrated the general utility of the pfMBP fusion system to express hemagglutinin fusion proteins.

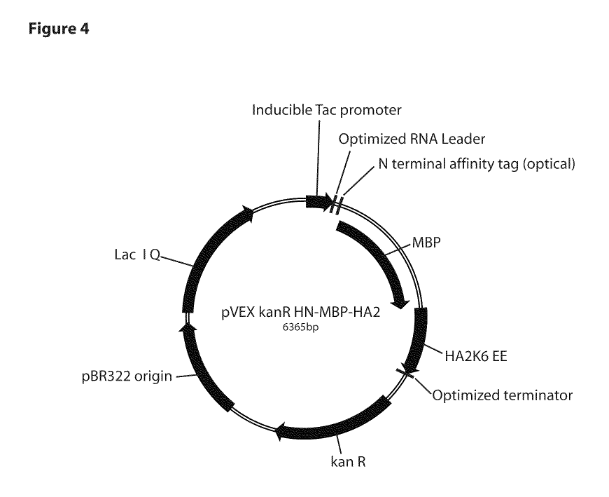

The HA2 gene was cloned in the pVEX kan HN MBP vector. The map (FIG. 4) and sequence of the resultant clone, pVEXkan HN MBP-HA2 is shown in SEQ ID NO: 3. High levels of thermostable fusion protein were induced after IPTG induction in large scale. This further demonstrated that addition of the 2.3 kd N terminal HN tag to pfMBP did not affect expression or thermostability of the fusion protein (1.7:1 MBP:target protein size ratio).

Thermostable fusion protein was purified from a 1 Liter IPTG-induced shake flask culture. Cells were: resuspended in binding buffer (50 mM NaPO₄, pH 7, 300 mM NaCl); lysed by freeze thawing and sonication; centrifuged; E. coli proteins were precipitated by heat treatment (75° C. for 40 minutes); and the clarified lysate (primarily a single HN-MBP-HA2 protein of the expected size) was purified on a Talon resin column, including a wash in binding buffer containing 0.1% Triton X-114, at 4° C. to reduce endotoxin. The final yield was 12 mg of high purity, intact, low endotoxin fusion protein (247 EU/mg). In contrast, HN-HA2 expressed from pVEXkan HN-HA2 (expression construct containing a precise deletion of the pfMBP gene) was insoluble. This demonstrated that the pVEXkan HN MBP-HA2 expression system can be utilized to produce high yields of high quality, soluble HA2 protein.

Collectively these results confirm the novel observation that pfMBP (43 kd) can stabilize target proteins much larger than the approximately 2:1 (thermostable fusion partner:target protein) size ratio, contrary to the teachings of de Marco et al, Supra, 2004. The results further demonstrated the general utility of pfMBP to generically stabilize fused target proteins. Moreover, pfMBP fusions purified by heat treatment (to flocculate and remove host E. coli proteins and thermolabile components) simplified the purification process.

### Example 4

### pfMBP-HA2 Fusion is an effective Immunogen

The immunogenicity of: 1) pfMBP-HA2 protein; and 2) a CMV promoter DNA vaccine plasmid expressing the entire influenza H5 vietnam 1203 04 serotype hemagglutinin (HA) gene (HA plasmid), were determined.

An amount of 10 μg of either HA plasmid DNA or pfMBP-HA2 protein (low endotoxin protein preparation from Example 3), mixed in 50 μL PBS pH7.4, was injected bilaterally (25 uL per site), intradermally into BALB/c mice (6-8 weeks old, 5 per group) in a prime dose (day 0). Boost doses were performed on Day 21 and 49. Terminal bleed was at 56 days.

Serum samples were taken at days 0, 21, 49 and 56 and tested for anti-HA2 IgG response. Anti-HA2 specific total IgG, IgG2a, and IgG1 levels were determined by standard ELISA assay using plates coated with Nature Technology Corporation's recombinant (E. coli) HN-HA2 protein (Example 3), which were purified under denaturing conditions (binding, wash, and elution buffers contained 4 M urea) on a Talon resin column. This antigen did not contain pfMBP. Sigma (St Louis, Mo.) detection antibodies, and the BD Biosciences OptEIA kit (BD Biosciences Pharmingen, San Diego, Calif.) reagents were used to develop ELISA plates. Briefly, coated wells were blocked and incubated 2 hrs with diluted serum samples. The wells were washed and the signal was detected by subsequent, sequential incubations and washing with either: 1) goat anti-mouse IgG (Fc)-biotin, then Streptavidin-horseradish peroxidase conjugate (total IgG); or 2) goat anti-mouse IgG1, then anti-goat IgG-biotin, then Streptavidin-horseradish peroxidase conjugate (IgG1); or 3) goat anti-mouse IgG2a, then anti-goat IgG-biotin, then Streptavidin-horseradish peroxidase conjugate (IgG1). The results, summarized in Table 2, demonstrated that pfMBP-HA2 was an effective immunogen, generating significantly more anti-HA2 total IgG (day 56, prime and two boosts) and IgG1 (day 49, prime-boost) antibodies than a comparator DNA immunogen. The DNA immunogen generated more anti-HA2 IgG2a antibodies than pfMBP-HA2 (day 49, prime-boost). This further demonstrated that the pfMBP-HA2 protein immunogen generated a Th2 biased response, compared to a DNA immunogen. Significantly, these results demonstrated that pfMBP fusion proteins can generate strong immune responses against a target antigen (HA2) at low dose, and in physiological buffers, without requiring additional adjuvant. Fusion to pfMBP enables use of the HA2 antigen as an immunogen since the native HA2 antigen with pfMBP (HN-HA2) was insoluble (Example 3) and unsuitable for use as an immunogen.

TABLE 2
pfMBP-HA2 is an effective immunogen

| | 21 day anti- | | | 49 day anti- | 56 day anti- |
|---|---|---|---|---|---|
| | HA2 IgG | | | HA2 IgG | HA2 IgG |
| | (total) | 49 day anti- | 49 day anti- | (total) | (total) |
| | (Abs | HA2 IgG1 | HA2 IgG2a | (Abs | (Abs |
| Immunogen | 1/62,500)** | (Abs 1/500)*** | (Abs 1/500)**** | 1/62,500)** | 1/62,500)** |
| pfMBP- | 0.073 ± 0.015 | 1.377 ± 0.08* | 0.395 ± 0.410 | 0.301 ± 0.102 | 0.615 ± 0.245* |
| HA2 | | | | | |
| HA plasmid | 0.073 ± 0.015 | 0.485 ± 0.468* | 0.904 ± 0.472 | 0.210 ± 0.154 | 0.190 ± 0.138* |

*P < .05 by two sided student t-test
**Total OD₄₅₀. T = 0 preimmune anti-HA2 IgG (total)(Abs 1/62,500) was 0.56 ± 0.002 (pfMBP-HA2) or 0.57 ± 0.002 (HA plasmid)
***Buffer blanked OD₄₅₀. T = 0 preimmune anti-HA2 IgG1(Abs 1/500) was 0.03 ± 0.03 (pfMBP-HA2) or 0.03 ± 0.03 (HA plasmid)
****Buffer blanked OD₄₅₀. T = 0 preimmune anti-HA2 IgG2a (Abs 1/500) was 0.003 ± 0.002 (pfMBP-HA2) or 0.001 ± 0.002 (HA plasmid)

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method to improve thermostability of a target protein molecule, the method comprising:
a. constructing a DNA expression vector which expresses a fusion protein in a transformed host cell, the fusion protein comprising a maltodextrin-binding domain and a target protein domain, the target protein domain having a molecular weight that is greater than 50% of the molecular weight of the maltodextrin-binding protein domain;
b. introducing the expression vector into a host cell;
c. allowing the host to express the fusion protein; and
d. recovering the said fusion protein.

2. The method of claim 1 wherein said expression vector is episomal or chromosomal.

3. The method of claim 1 wherein the maltodextrin-binding domain comprises an amino acid sequence at least 95% identical to the sequence of SEQ ID NO: 4.

4. A fusion protein comprising:
a maltodextrin-binding protein domain;
a target protein domain having a molecular weight that is greater than 50% of the molecular weight of the maltodextrin-binding protein domain.

5. The fusion protein of claim 4 wherein the maltodextrin-binding domain comprises an amino acid sequence at least 95% identical to the sequence of SEQ ID NO: 4.

6. The fusion protein of claim 4 wherein the target protein domain is derived from a protein selected from the group consisting of: viral pathogen antigens; bacterial pathogen antigens; influenza antigens; influenza hemagglutinin; influenza hemagglutinin HA1 domain; influenza hemagglutinin HA2 domain; protein adjuvants; and flagellin.

7. A method to produce a fusion protein having a targeted antigenic domain, the method comprising:
a. constructing a DNA expression vector which expresses a fusion protein in a transformed host cell, the fusion protein comprising a maltodextrin-binding protein with at least 95% sequence identity to the sequence set forth as SEQ ID NO: 4 and a target antigenic protein domain;
b. introducing said expression vector into a host cell;
c. expressing the said fusion protein; and
d. recovering the said fusion protein, wherein the fusion protein has sufficient solubility to elicit an immunological response in an animal.

8. The method of claim 7 wherein said expression vector is episomal or chromosomal.

9. A fusion protein antigen comprising:
a. a maltodextrin-binding protein with at least 95% sequence identity to the sequence set forth as SEQ ID NO: 4, operably linked to,
b. a target antigenic protein domain,
wherein the fusion protein has sufficient solubility to elicit an immunological response in an animal.

10. The fusion protein of claim 9 wherein the target antigenic protein domain consists of a domain that is at least 90% identical to a protein selected from the group consisting of: viral pathogen antigens; bacterial pathogen antigens; influenza antigens; influenza hemagglutinin; influenza hemagglutinin HA1 domain; and influenza hemagglutinin HA2 domain.

11. A fusion protein comprising:
a. a maltodextrin-binding protein with at least 95% sequence identity to the sequence set forth as SEQ ID NO: 4;
b. a protein adjuvant domain;
wherein the said fusion protein has a greater thermostability than the protein consisting only of the protein adjuvant domain.

12. The fusion protein of claim 11 wherein the protein adjuvant domain consists of a protein domain that is derived from a protein selected from the group consisting of: toll-like receptor agonist; complement receptor agonist; flagellin; salmonella flagellin; porin; Neisserial porin; Neisseria meningitidis PorB; type 1 fimbria fimH adhesion protein; cholera ADP-ribosylating enterotoxin; Escherichia coli heat labile enterotoxin; HSP60; cytokines; chemokines; and Toxoplasma gondii profilin.
